(19) United States
(12) Patent Application Publication
(10) Pub. No.: US 2026/0182907 A1
(43) Pub. Date: Jul. 2, 2026

(54) SYSTEM AND METHOD FOR DETERMINING SLEEP STATE USING BIOLOGICAL ACTIVITY DOPPLER SIGNAL

(71) Applicant: JCF TECHNOLOGY INC., Seoul (KR)

(72) Inventor: Jin Myung KIM, Bucheon-si, Gyeonggi-do (KR)

(21) Appl. No.: 18/840,912

(22) PCT Filed: Apr. 10, 2024
(86) PCT No.: PCT/KR2024/004783

§ 371 Date: Aug. 23, 2024

(30) Foreign Application Priority Data
Apr. 11, 2023 (KR) 10-2023-0047855

(51) Int. Cl. A61B 5/00; A61B 5/0205; A61B 5/024; A61B 5/05; A61B 5/08

(52) U.S. Cl. CPC A61B 5/4812; A61B 5/0205; A61B 5/024; A61B 5/05; A61B 5/0803; A61B 5/4818; A61B 5/7257; A61B 5/7264; A61B 5/7271

(57) ABSTRACT

Provided is a system and method for determining a sleep state using a biological activity Doppler signal which comprises: a Doppler signal acquisition unit for acquiring a Doppler signal including biological activity information using a radar; an auxiliary signal processing unit for acquiring a tossing and turning motion and a snoring noise as auxiliary signals using a thermal image sensor and a noise sensor; a Doppler signal analysis unit for acquiring spectrum energy at preset intervals by analyzing the Doppler signal, determining whether the spectrum energy is acquired periodically, and performing classification of the spectrum energy using the auxiliary signals; and a sleep-stage definition unit for defining a sleep state of each stage in an entire sleep stage using a combination of a ratio of respiration spectrum energy and heartbeat spectrum energy, among the spectrum energy, and the non-periodic spectrum energy.

### TECHNICAL FIELD

The present invention relates to a system and method for determining a sleep state using a biological activity Doppler signal, and particularly, to a system and method for determining a sleep state using a biological activity Doppler signal, which can acquire a Doppler signal of a user's biological activity in a sleep state of the user using a radar, and analyze the sleep state of the user by analyzing the acquired Doppler signal.

### BACKGROUND ART

Recently, the number of people in need of care or attention is increasing in the domestic society. The people in need of care or attention may be defined as people who need assistance when an emergency situation occurs or people who do not have a housemate, and for example, the elderly, the disabled, and single-person households may be included. According to a recent survey, the elderly population has exceeded 9 million, and the population of single-person households and registered disabled people has also exceeded 6.6 million and 2.6 million, respectively. In addition, the number of people who die alone due to absence of a housemate exceeds 10,000.

In addition, when such single-person households or the like lead to irregular lives, quality of sleep comes to be irregular, and this may impair health in some cases. In order to measure the quality of sleep, it needs to go through a troublesome process of making an appointment at a specialized hospital, visiting the hospital on time, and using specialized devices while sleeping, and therefore, there is a problem in that it is difficult to measure the quality of sleep regularly.

### DISCLOSURE OF INVENTION

### Technical Problem

To solve the problems of the prior art as described above, an embodiment of the present invention provides a system and method for determining a sleep state using a biological activity Doppler signal, which can acquire biological activity information in a sleep state of a user using a radar, and define the sleep state of the user by performing an analysis on the acquired biological activity information.

### Technical Solution

To accomplish the above object, according to one aspect of the present invention, there is provided a sleep state determination system using a biological activity Doppler signal. The sleep state determination system using a biological activity Doppler signal comprises: a Doppler signal acquisition unit for acquiring a Doppler signal including biological activity information using a radar;
a Doppler signal analysis unit for acquiring spectrum energy at preset intervals by analyzing the Doppler signal, and determining whether the spectrum energy is acquired periodically; and
a sleep-stage definition unit for defining a sleep state of each stage in an entire sleep stage using a combination of a ratio of respiration spectrum energy and heartbeat spectrum energy, among the spectrum energy, and the non-periodic spectrum energy.

The Doppler signal may include a respiration Doppler signal that acquires information on breathing of a user and a heartbeat Doppler signal that acquires information on heartbeat of the user.

The Doppler signal analysis unit may acquire the spectrum energy by performing a fast Fourier transform on the Doppler signal.

The sleep-stage definition unit may define a stage in which the non-periodic spectrum energy does not exist, among stages where the ratio of respiration spectrum energy and heartbeat spectrum energy is 5:5 or higher in the entire sleep stage, as a deep sleep stage, and define a stage in which the non-periodic spectrum energy does not exist, among stages where the ratio of respiration spectrum energy and heartbeat spectrum energy is lower than 5:5, as an apnea stage.

The sleep-stage definition unit may define a stage in which the non-periodic spectrum energy exists and the non-periodic spectrum energy appears to be higher than or equal to a preset magnitude as a tossing and turning stage, and define a stage in which the non-periodic spectrum energy appears to be lower than or equal to a preset magnitude in the apnea stage as a snoring stage.

According to an aspect of the present invention, there is provided a sleep state determination system using a biological activity Doppler signal. The sleep state determination system using a biological activity Doppler signal comprises: a Doppler signal acquisition unit for acquiring a Doppler signal including biological activity information using a radar; a Doppler signal analysis unit for acquiring spectrum energy at preset intervals by analyzing the Doppler signal, and determining whether the spectrum energy is acquired periodically; and a sleep-stage definition unit for defining sleep states of the other sleep stages using a preset ratio range and the non-periodic spectrum energy on the basis of an average of the spectrum energy of a sleep entry stage that satisfies preset criteria in an entire sleep stage.

According to an aspect of the present invention, there is provided a sleep state determination method using a biological activity Doppler signal. The sleep state determination method using a biological activity Doppler signal comprises: a Doppler signal acquisition step of acquiring, by a Doppler signal acquisition unit, a Doppler signal including biological activity information using a radar;
a Doppler signal analysis step of acquiring spectrum energy at preset intervals by analyzing the Doppler signal, and analyzing the Doppler signal by determining whether the spectrum energy is acquired periodically; and
a sleep-stage definition step of defining, by a sleep-stage definition unit, a sleep state of each stage in an entire sleep stage using a combination of a ratio of respiration spectrum energy and heartbeat spectrum energy, among the spectrum energy, and the non-periodic spectrum energy.

The Doppler signal may include a respiration Doppler signal that acquires information on breathing of a user and a heartbeat Doppler signal that acquires information on heartbeat of the user.

The Doppler signal analysis step may acquire the spectrum energy by performing a fast Fourier transform on the Doppler signal.

The sleep-stage definition step may define a stage in which the non-periodic spectrum energy does not exist, among stages where the ratio of respiration spectrum energy and heartbeat spectrum energy is 5:5 or higher in the entire sleep stage, as a deep sleep stage, and define a stage in which the non-periodic spectrum energy does not exist, among stages where the ratio of respiration spectrum energy and heartbeat spectrum energy is lower than 5:5, as an apnea stage.

The sleep-stage definition step may define a stage in which the non-periodic spectrum energy exists and the non-periodic spectrum energy appears to be higher than or equal to a preset magnitude as a tossing and turning stage, and define a stage in which the non-periodic spectrum energy appears to be lower than or equal to a preset magnitude in the apnea stage as a snoring stage.

According to an aspect of the present invention, there is provided a sleep state determination method using a biological activity Doppler signal. The sleep state determination method using a biological activity Doppler signal comprises the steps of: acquiring, by a Doppler signal acquisition unit, a Doppler signal including biological activity information using a radar; acquiring, by a Doppler signal analysis unit, spectrum energy at preset intervals by analyzing the Doppler signal, and determining whether the spectrum energy is acquired periodically; and defining, by a sleep-stage definition unit, sleep states of the other sleep stages using a preset ratio range and the non-periodic spectrum energy on the basis of an average of the spectrum energy of a sleep entry stage that satisfies preset criteria in an entire sleep stage.

### Advantageous Effects

The system and method for determining a sleep state using a biological activity Doppler signal according to an embodiment of the present invention has an effect of not disturbing sleep of a user as it does not affect the user's body by acquiring biological activity information using a radar while the user sleeps.

In addition, the system and method for determining a sleep state using a biological activity Doppler signal according to an embodiment of the present invention has an effect of accurately determining a sleep state of a user in each sleep stage using breathing and heartbeat information of the user.

### BRIEF DESCRIPTION OF THE DRAWINGS

FIG. 1 is a block diagram showing a sleep state determination system using a biological activity Doppler signal according to an embodiment of the present invention.

FIG. 2 is a flowchart illustrating a sleep state determination method using a biological activity Doppler signal according to an embodiment of the present invention.

FIG. 3 is a graph showing a result of an actual simulation using an embodiment of the present invention.

### BEST MODE FOR CARRYING OUT THE INVENTION

Hereinafter, some embodiments of the present disclosure will be described in detail with reference to illustrative drawings. In adding reference numerals to components in each drawing, like components may have like reference numerals as much as possible although they are shown in different drawings. In addition, in describing the present embodiments, when it is determined that a detailed description of a related known configuration or function may obscure the gist of the present technical spirit, the detailed description may be omitted. When “comprise”, “have”, “configured of”, and the like mentioned in the specification are used, other parts may be added as long as “only” is not used. When a component is expressed in a singular form, it may also include the plural, unless specifically stated otherwise.

In addition, in describing the components of the present disclosure, terms such as first, second, A, B, (a), and (b) may be used. These terms are used only to distinguish the components from other components, and the nature, sequence, order, or number of the components are not limited by the terms.

When two or more components are described as being “connected”, “coupled”, or “combined” in describing the positional relationship of components, although two or more components may be directly “connected”, “coupled”, or “combined”, it should be understood that the two or more components are “connected”, “coupled”, or “combined” as other components are further “interposed”. Here, other components may be included in one or more of two or more components “connected”, “coupled”, or “combined” with each other.

In describing the relationship of temporal flows related to the components, operation methods, manufacturing methods, and the like, for example, when a temporal precedence relationship or a flow precedence relationship is described as “after”, “in succession to”, “next”, “before”, or the like, non-successive cases may also be included unless “immediately” or “directly” is used.

Meanwhile, when a numerical value about a component or information corresponding thereto (e.g., levels, etc.) is mentioned, although there is no explicit description separately, the numerical value or information corresponding thereto may be interpreted as including a range of error that may occur due to various factors (e.g., processing factors, internal or external shocks, noise, and the like).

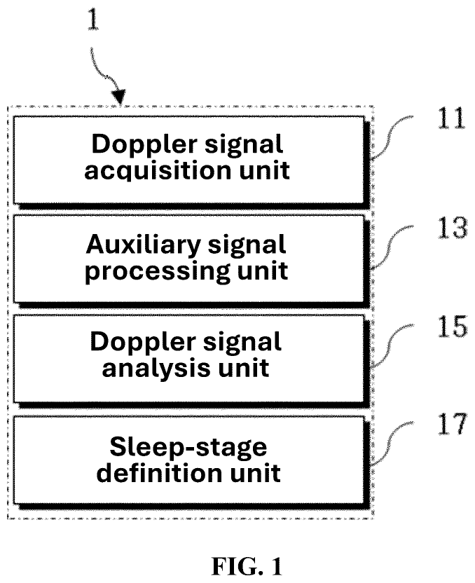

FIG. 1 is a block diagram showing a sleep state determination system using a biological activity Doppler signal according to an embodiment of the present invention. A sleep state determination system (1) using a biological activity Doppler signal according to an embodiment of the present invention may be formed to be installed in a space where a user lives and sleeps, such as a master bedroom or the like, to detect sleep when the user falls asleep, acquire Doppler signals for biological activities, and define a sleep state of the user by analyzing the acquired Doppler signals. To this end, the present invention may use a biological activity measurement device using a radar previously installed in the space described above. As shown in FIG. 1, the sleep state determination system (1) using a biological activity Doppler signal according to an embodiment of the present invention may be formed to include a Doppler signal acquisition unit (11), an auxiliary signal processing unit (13), a Doppler signal analysis unit (15), and a sleep-stage definition unit (17).

The Doppler signal acquisition unit (11) is formed to acquire a Doppler signal including biological activity information from a radar installed in a user's sleeping space. The Doppler signal is formed to include a respiration Doppler signal, which is information on breathing of the user, and a heartbeat Doppler signal, which is information on heartbeat of the user. The Doppler signal may be defined as a signal that includes a Doppler frequency generated by movement of the user when the radar transmits radio waves to the user and the transmitted radio waves return. In an embodiment of the present invention, the respiration Doppler signal and the heartbeat Doppler signal may be Doppler signals with respect to the change in the movement of body organs that move while breathing and body organs that move while the heart is beating.

The auxiliary signal processing unit (13) is formed to acquire auxiliary signals using an auxiliary biological activity information acquisition device further provided in the user's sleeping space, and acquire information on the movement (tossing and turning, or the like) of body parts, other than breathing or heartbeat, of the user, noise, or the like by analyzing the acquired auxiliary signals. To this end, the auxiliary signal processing unit (13) may acquire measurement results from a thermal image sensor and a noise measurement sensor, which are auxiliary biological activity information acquisition devices. The thermal image sensor may be provided to measure movement of the body of the user, and the noise measurement sensor may be provided to measure the snoring sound of the user.

The auxiliary signal processing unit (13) may continuously acquire contour information of the user's body using the thermal image sensor. When contour information of the user's body is continuously acquired using the thermal image sensor, changes in the contour information generated due to the movement, such as tossing and turning or the like, of the user can be acquired.

In addition, the auxiliary signal processing unit (13) may measure noise generated due to snoring of the user using the noise measurement sensor and confirm the noise as an auxiliary index for determining whether the user is snoring currently.

The body contour information and noise information acquired by the auxiliary signal processing unit (13) may be used by the Doppler signal analysis unit (15) described below, and as an example, when the Doppler signal analysis unit (15) acquires non-periodic spectrum energy using information on the time of acquiring tossing and turning motions or snoring motions expressed as non-periodic spectrum energy, the non-periodic spectrum energy may be used as auxiliary information for determining what kind of biological activity information the energy means.

The Doppler signal analysis unit (15) is formed to acquire spectrum energy at preset intervals by analyzing the Doppler signals acquired through the Doppler signal acquisition unit (11). The Doppler signal analysis unit (15) may acquire spectrum energy by applying a preset function to the Doppler signals, and acquire respiration spectrum energy and heartbeat spectrum energy, which are spectrum energies of the respiration Doppler signal and the heartbeat Doppler signal, by analyzing the acquired spectrum energy according to preset criteria.

In an embodiment of the present invention, the Doppler signal analysis unit (15) may use a fast Fourier transform (FFT) as the preset function used to acquire spectrum energy from the Doppler signals at preset intervals. The Fourier transform is a well-known function and means a transformation that decomposes signals sampled in time or space into components of time frequency or space frequency. When the Fourier transform is used, the Doppler signal analysis unit (15) may convert the Doppler signals acquired at preset intervals into spectrum energy of a specific frequency component.

The Doppler signal analysis unit (15) according to an embodiment of the present invention is formed to confirm, when the spectrum energy of a specific frequency component is acquired, whether the acquired spectrum energy of a specific frequency component is spectrum energy generated periodically. The Doppler signal analysis unit (15) may be formed to continuously acquire spectrum energy of the Doppler signals acquired at preset intervals and determine whether the acquired continuous spectrum energy has periodicity. Here, when the spectrum energy has periodicity, the spectrum energy may be determined as spectrum energy of breathing or heartbeat, and when the spectrum energy has no periodicity, the spectrum energy may not be determined as spectrum energy of breathing or heartbeat.

The Doppler signal analysis unit (15) may confirm whether the non-periodic spectrum energy has an energy higher than a preset magnitude, and when it has an energy higher than a preset magnitude, the spectrum energy may be determined as spectrum energy of the Doppler frequency generated due to body movement of the user. In addition, it may be formed to determine, when the spectrum energy has an energy lower than a preset magnitude, the spectrum energy as spectrum energy of the Doppler frequency generated due to snoring of the user.

In addition, the Doppler signal analysis unit (15) according to an embodiment of the present invention may perform analysis on periodic spectrum energy using a preset determination algorithm, and separately acquire respiration spectrum energy and heartbeat spectrum energy as a result of the analysis.

In addition, the Doppler signal analysis unit (15) according to an embodiment of the present invention may identify the type of the non-periodic spectrum energy using the auxiliary signal acquired by the auxiliary signal processing unit (13) described above. Here, the Doppler signal analysis unit (15) may be formed to acquire non-periodic spectrum energy of tossing and turning (body movement) of the user using, for example, thermal image information, and determine the acquired non-periodic spectrum energy as a noise signal and remove it from data for analyzing sleep stages described below.

In addition, in another embodiment, when an energy of a preset magnitude that can distinguish body movement from snoring using the non-periodic spectrum energy is not defined, the Doppler signal analysis unit (15) may define an energy of a preset magnitude that can distinguish the two movements using the auxiliary signals acquired through a noise sensor and a thermal image sensor.

The sleep-stage definition unit (17) is formed to define, when the Doppler signal analysis unit (15) according to an embodiment of the present invention continuously acquire spectrum energy of a specific frequency component, and determining whether or not the spectrum energy periodically occurs is completed, a sleep state in each stage of the entire sleep stage using an analysis result.

The entire sleep stage may include at least one among a sleep entry stage, a deep sleep stage, a tossing and turning stage, an apnea stage, and a snoring stage. The sleep entry stage means a stage in which the user enters sleep, and this is a stage in which whether the user begins to sleep can be determined, which occurs statistically in almost all users. In addition, the deep sleep stage means a sleep stage in which physical activities of the user are maintained stably, while the tossing and turning stage means a sleep stage in which the physical activities of the user include movement. In addition, the apnea stage means a sleep stage in which breathing activity does not occur during the physical activities of the user, and the snoring stage means a sleep stage in which snoring occurs during the physical activities of the user.

The higher the proportion of the deep sleep occupied in the sleep stage and the lower the proportion of the other stages, the higher the quality of the sleep. Therefore, when each sleep stage can be defined through the present invention, information on the sleep quality of the user can be acquired, and therefore, a prescription corresponding to the sleep quality can be provided for each user at a later time.

The sleep-stage definition unit (17) is formed to define a sleep state of each stage in the entire sleep stage using an analysis result of the Doppler signal analysis unit (15). Each stage in the entire sleep stage includes at least one among the sleep entry stage, deep sleep stage, tossing and turning stage, apnea stage, and snoring stage as described above. The sleep-stage definition unit (17) acquires an analysis result from the Doppler signal analysis unit (15) and determines which sleep stage the analysis result corresponds to using preset state determination criteria.

The preset state determination criteria may be a value set in advance using the ratio of the respiration spectrum energy and the heartbeat spectrum energy acquired by the Doppler signal analysis unit (15). As described above, since the magnitude of physical activities generating during the breathing motion is generally higher than the magnitude of physical activities generating during the heartbeat motion, the magnitude of the respiration spectrum energy appears to be higher than the magnitude of the heartbeat spectrum energy in a stable sleep stage. In addition, in the snoring stage, as high frequency vibration is captured, an additional spectrum other than the respiration spectrum energy and the heartbeat spectrum energy may appear. In addition, in the apnea stage, since a breathing motion does not occur or slightly occurs, the magnitude of the respiration spectrum energy may decrease as low as the magnitude of the heartbeat spectrum energy.

The sleep-stage definition unit (17) of an embodiment of the present invention may define a stage in which the spectrum ratio, which is the ratio of the respiration spectrum energy to the heartbeat spectrum energy, is 5:5 or higher as a deep sleep stage.

In addition, the sleep-stage definition unit (17) may define a stage in which the non-periodic spectrum energy is acquired to be lower than a preset magnitude, among the stages in which the spectrum ratio is lower than 5:5, as a snoring stage, and when the non-periodic spectrum energy is not acquired for a predetermined period of time, the stage may be defined as an apnea stage.

In another embodiment of the present invention, the sleep-stage definition unit (17) may define a sleep stage using the sleep entry stage, in addition to the spectrum ratio. Since it is general that the tossing and turning, apnea, or snoring does not occur in the sleep entry stage like in the deep sleep stage, the sleep-stage definition unit (17) of the present invention may be formed to acquire a sleep entry stage first and then determine a deep sleep stage based on the acquired sleep entry stage.

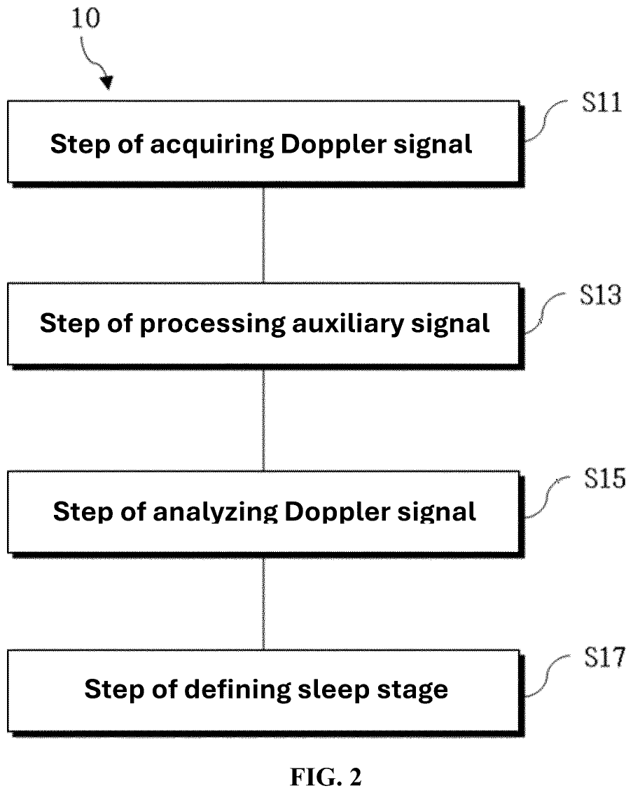

Meanwhile, FIG. 2 shows a flowchart illustrating a sleep state determination method using a biological activity Doppler signal according to an embodiment of the present invention. Hereinafter, although it is described that a sleep state determination method using a biological activity Doppler signal of the present invention is performed using the system of FIG. 1 for convenience of explanation, the present invention is not necessarily limited thereto.

A sleep state determination method (10) using a biological activity Doppler signal according to an embodiment of the present invention may be formed to be installed in a space where a user lives and sleeps, such as a master bedroom or the like, to detect sleep when the user falls asleep, acquire Doppler signals for biological activities, and define a sleep state of the user by analyzing the acquired Doppler signals. To this end, the present invention may use a biological activity measurement device using a radar previously installed in the space described above. As shown in FIG. 2, the sleep state determination method (10) using a biological activity Doppler signal according to an embodiment of the present invention may be formed to include a step of acquiring a Doppler signal (S11), a step of processing an auxiliary signal (S13), a step of analyzing the Doppler signal (S15), and a step of defining a sleep stage (S17).

The step of acquiring a Doppler signal (S11) is formed to acquire, by the Doppler signal acquisition unit, a Doppler signal including biological activity information from a radar installed in a user's sleeping space. The Doppler signal is formed to include a respiration Doppler signal, which is information on breathing of the user, and a heartbeat Doppler signal, which is information on heartbeat of the user. The Doppler signal may be defined as a signal that includes a Doppler frequency generated by movement of the user when the radar transmits radio waves to the user and the transmitted radio waves return. In an embodiment of the present invention, the respiration Doppler signal and the heartbeat Doppler signal may be Doppler signals with respect to the change in the movement of body organs that move while breathing and body organs that move while the heart is beating.

The step of processing an auxiliary signal (S13) is formed to acquire auxiliary signals using an auxiliary biological activity information acquisition device further provided in the user's sleeping space, and acquire information on the movement (tossing and turning, or the like) of body parts other than breathing or heartbeat, noise, or the like of the user by analyzing the acquired auxiliary signals. To this end, the step of processing an auxiliary signal (S13) may acquire measurement results from a thermal image sensor and a noise measurement sensor, which are auxiliary biological activity information acquisition devices. The thermal image sensor may be provided to measure the movement of the body of the user, and the noise measurement sensor may be provided to measure the snoring sound of the user.

The step of processing an auxiliary signal (S13) may continuously acquire contour information of the user's body using the thermal image sensor. When contour information of the user's body is continuously acquired using the thermal image sensor, changes in the contour information generated due to the movement, such as tossing and turning or the like, of the user can be acquired.

In addition, the step of processing an auxiliary signal (S13) may measure noise generated due to snoring of the user using the noise measurement sensor and confirm the noise as an auxiliary index for determining whether the user is snoring currently.

The body contour information and noise information acquired at the step of processing an auxiliary signal (S13) may be used at the step of analyzing the Doppler signal (S15) described below, and as an example, when the step of analyzing the Doppler signal (S15) acquires non-periodic spectrum energy using information on the time of acquiring tossing and turning motions or snoring motions expressed as non-periodic spectrum energy, the non-periodic spectrum energy may be used as auxiliary information for determining what kind of biological activity information the energy means.

The step of analyzing the Doppler signal (S15) is formed to acquire spectrum energy at preset intervals by analyzing, by the Doppler signal analysis unit, the Doppler signals acquired through the step of acquiring a Doppler signal (S11). The step of analyzing the Doppler signal (S15) may acquire spectrum energy by applying a preset function to the Doppler signals, and acquire respiration spectrum energy and heartbeat spectrum energy, which are spectrum energies of the respiration Doppler signal and the heartbeat Doppler signal, by analyzing the acquired spectrum energy according to preset criteria.

In an embodiment of the present invention, the step of analyzing the Doppler signal (S15) may use a fast Fourier transform (FFT) as the preset function used to acquire spectrum energy from the Doppler signals at preset intervals. The Fourier transform is a well-known function and means a transformation that decomposes signals sampled in time or space into components of time frequency or space frequency. When the Fourier transform is used, the step of analyzing the Doppler signal (S15) may convert the Doppler signals acquired at preset intervals into spectrum energy of a specific frequency component.

The step of analyzing the Doppler signal (S15) according to an embodiment of the present invention is formed to confirm, when the spectrum energy of a specific frequency component is acquired, whether the acquired spectrum energy of a specific frequency component is spectrum energy generated periodically. The step of analyzing the Doppler signal (S15) may be formed to continuously acquire spectrum energy of the Doppler signals acquired at preset intervals and determine whether the acquired continuous spectrum energy has periodicity. Here, when the spectrum energy has periodicity, the spectrum energy may be determined as spectrum energy of breathing or heartbeat, and when the spectrum energy has no periodicity, the spectrum energy may not be determined as spectrum energy of breathing or heartbeat.

The step of analyzing the Doppler signal (S15) may confirm whether the non-periodic spectrum energy has an energy higher than a preset magnitude, and when it has an energy higher than a preset magnitude, the spectrum energy may be determined as spectrum energy of the Doppler frequency generated due to body movement of the user. In addition, it may be formed to determine, when the spectrum energy has an energy lower than a preset magnitude, the spectrum energy as spectrum energy of the Doppler frequency generated due to snoring of the user.

In addition, the step of analyzing the Doppler signal (S15) according to an embodiment of the present invention may perform analysis on periodic spectrum energy using a preset determination algorithm, and separately acquire respiration spectrum energy and heartbeat spectrum energy as a result of the analysis.

In addition, the step of analyzing the Doppler signal (S15) according to an embodiment of the present invention may identify the type of the non-periodic spectrum energy using the auxiliary signal acquired at the step of processing an auxiliary signal (S13) described above. Here, the step of analyzing the Doppler signal (S15) may be formed to acquire non-periodic spectrum energy of tossing and turning (body movement) of the user using, for example, thermal image information, and determine the acquired non-periodic spectrum energy as a noise signal and remove it from data for analyzing sleep stages described below.

In addition, in another embodiment, when an energy of a preset magnitude that can distinguish body movement from snoring using the non-periodic spectrum energy is not defined, the step of analyzing the Doppler signal (S15) may define an energy of a preset magnitude that can distinguish the two movements using the auxiliary signals acquired through a noise sensor and a thermal image sensor.

The step of defining a sleep stage (S17) is formed to define, when the step of analyzing the Doppler signal (S15) according to an embodiment of the present invention continuously acquire spectrum energy of a specific frequency component, and determining whether or not the spectrum energy periodically occurs is completed, a sleep state in each stage of the entire sleep stage using an analysis result.

The entire sleep stage may include at least one among a sleep entry stage, a deep sleep stage, a tossing and turning stage, an apnea stage, and a snoring stage. The sleep entry stage means a stage in which the user enters sleep, and this is a stage in which whether the user begins to sleep can be determined, which occurs statistically in almost all users. In addition, the deep sleep stage means a sleep stage in which physical activities of the user are maintained stably, while the tossing and turning stage means a sleep stage in which the physical activities of the user include movement. In addition, the apnea stage means a sleep stage in which breathing activity does not occur during the physical activities of the user, and the snoring stage means a sleep stage in which snoring occurs during the physical activities of the user.

The higher the proportion of the deep sleep occupied in the sleep stage and the lower the proportion of the other stages, the higher the quality of the sleep. Therefore, when each sleep stage can be defined through the present invention, information on the sleep quality of the user can be acquired, and therefore, a prescription corresponding to the sleep quality can be provided for each user at a later time.

The step of defining a sleep stage (S17) is formed to define, by the sleep-stage definition unit, a sleep state of each stage in the entire sleep stage using an analysis result of the step of analyzing the Doppler signal (S15). Each stage in the entire sleep stage includes at least one among the sleep entry stage, deep sleep stage, tossing and turning stage, apnea stage, and snoring stage as described above. The step of defining a sleep stage (S17) acquires an analysis result from the step of analyzing the Doppler signal (S15) and determines which sleep stage the analysis result corresponds to using preset state determination criteria.

The preset state determination criteria may be a value set in advance using the ratio of the respiration spectrum energy and the heartbeat spectrum energy acquired at the step of analyzing the Doppler signal (S15). As described above, since the magnitude of physical activities generating during the breathing motion is generally higher than the magnitude of physical activities generating during the heartbeat motion, the magnitude of the respiration spectrum energy appears to be higher than the magnitude of the heartbeat spectrum energy in a stable sleep stage. In addition, in the snoring stage, as high frequency vibration is captured, an additional spectrum other than the respiration spectrum energy and the heartbeat spectrum energy may appear. In addition, in the apnea stage, since a breathing motion does not occur or slightly occurs, the magnitude of the respiration spectrum energy may decrease as low as the magnitude of the heartbeat spectrum energy.

The step of defining a sleep stage (S17) of an embodiment of the present invention may define a stage in which the spectrum ratio, which is the ratio of the respiration spectrum energy to the heartbeat spectrum energy, is 5:5 or higher as a deep sleep stage.

In addition, the step of defining a sleep stage (S17) may define a stage in which the non-periodic spectrum energy is acquired to be lower than a preset magnitude, among the stages in which the spectrum ratio is lower than 5:5, as a snoring stage, and when the non-periodic spectrum energy is not acquired for a predetermined period of time, the stage may be defined as an apnea stage.

In another embodiment of the present invention, the step of defining a sleep stage (S17) may define a sleep stage using the sleep entry stage, in addition to the spectrum ratio. Since it is general that the tossing and turning, apnea, or snoring does not occur in the sleep entry stage like in the deep sleep stage, the step of defining a sleep stage (S17) of the present invention may be formed to acquire a sleep entry stage first and then determine a deep sleep stage based on the acquired sleep entry stage.

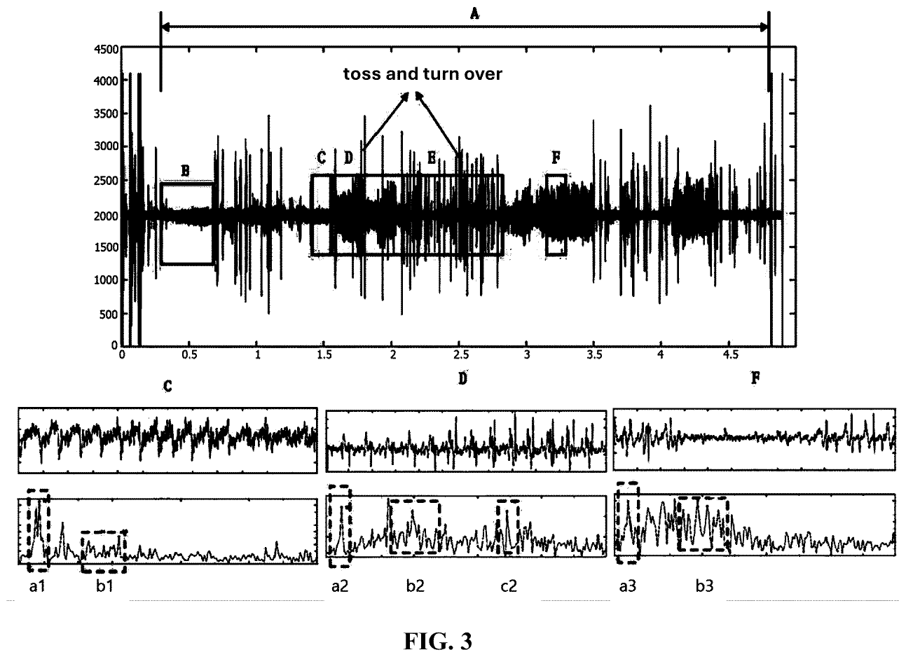

Meanwhile, FIG. 3 is a graph showing a result of an actual simulation using an embodiment of the present invention. Referring to FIG. 3, the entire sleep stage may be represented as A. Stage B is the sleep entry stage, which is a stage that occurs statistically when almost all users enter sleep as described above, and it may be a stage in which sleep of a user actually begins, which is a stage of 10 to 40 minutes, appearing as 20 to 30 minutes in average. Stage C is a deep sleep stage, which is a stable sleep stage, D is a snoring stage, E is a tossing and turning stage, and F is an apnea stage.

Stage C is a stable deep sleep stage, in which the magnitude of the respiration spectrum energy (a1) appears to be sufficiently higher than the magnitude of the heartbeat spectrum energy (b1). In addition, observing the periodicity of the spectrum energy, it can be confirmed that the spectrum energy does not include irregular peaks and repeats at a regular cycle. Therefore, in an embodiment of the present invention, this sleep stage may be defined as a deep sleep stage. At this point, in the present invention, as described above, a stage in which the ratio of respiration spectrum energy (a1) and heartbeat spectrum energy (b1) is 5:5 or higher may be defined as a deep sleep stage, and in another embodiment, a stage having a periodicity greater than or equal to a preset similarity and an energy lower than or equal to a preset magnitude compared to the sleep entry stage, i.e., stage B, may be defined as a deep sleep stage.

Stage D is a snoring stage, which is a stage in which although the magnitude of the respiration spectrum energy (a2) is formed to be higher than the magnitude of the heartbeat spectrum (b2), snoring spectrum energy (c2), which does not appear in the other stages, is additionally measured. In stage D, it can be confirmed that a spectrum energy like c2 is measured at a frequency higher than the frequency of breathing or heartbeat. Therefore, the sleep-stage definition unit and the step of defining a sleep stage of the present invention may define stage D as a snoring stage, in which the user is snoring.

Stage F is an apnea stage, in which the magnitude of the respiration spectrum energy (a3) appears to be similar to the magnitude of the heartbeat spectrum energy (b3). More specifically, this is a stage in which the ratio of the respiration spectrum energy (a) and the heartbeat spectrum energy (b3) appears to be lower than 5:5, and the snoring spectrum energy (c2) is not measured, unlike stage D. This is since that as the breathing activity in the body of the user is weak or does not exist in the process of acquiring a Doppler signal using a radar, the magnitude of the acquired respiration spectrum energy (a3) is reduced greatly. Therefore, like the spectrum analysis result for stage F of FIG. 3, when the magnitude of the respiration spectrum energy (a3) is compared to the magnitude of the heartbeat spectrum energy (b3), and the ratio is lower than 5:5, the sleep-stage definition unit and the step of defining a sleep stage according to an embodiment of the present invention may define stage F as an apnea stage.

Stage E is a stage in which tossing and turning occurs, which is a stage in which spectrum energy having an energy of high magnitude compared to the spectrum energy detected in stages B, C, D, and Fis acquired. This is generation of a biological activity having a very high energy compared to the respiration spectrum energy or the heartbeat spectrum energy, and the present invention is formed to analyze such a biological activity as tossing and turning.

In summary, the system and method of FIGS. 1 and 2 are formed to acquire and analyze Doppler signals of biological activities of a user using a radar, and define a sleep state of each sleep stage using respiration spectrum energy and heartbeat spectrum energy of the Doppler signals, and results of experiments on the sleep states are shown in FIG. 3. As the present invention described in FIGS. 1 to 3 is used, there is an effect of confirming the sleep state of a user and responding to generation of an emergency situation or providing contents for improving sleep quality of the user using the confirmed sleep state.

Although an embodiment of the present invention has been described above, the spirit of the present invention is not limited to the embodiment presented in the present specification, and although those skilled in the art who understand the spirit of the present invention may easily suggest other embodiments by adding, changing, deleting, or supplementing components within the scope of the same spirit, it will be said that this is also within the scope of the present invention.

1. A sleep state determination system using a biological activity Doppler signal, the system comprising:
a Doppler signal acquisition unit for acquiring a Doppler signal including biological activity information using a radar;
an auxiliary signal processing unit for acquiring a tossing and turning motion and a snoring noise as auxiliary signals using a thermal image sensor and a noise sensor;
a Doppler signal analysis unit for acquiring spectrum energy at preset intervals by analyzing the Doppler signal, determining whether the spectrum energy is acquired periodically, and performing, when the spectrum energy is determined as non-periodic spectrum energy, classification of the non-periodic spectrum energy using the auxiliary signals; and
a sleep-stage definition unit for defining a sleep state of each stage in an entire sleep stage using a combination of a ratio of respiration spectrum energy and heartbeat spectrum energy, among the spectrum energy, and the non-periodic spectrum energy.

2. The system according to claim 1, wherein the Doppler signal includes a respiration Doppler signal that acquires information on breathing of a user and a heartbeat Doppler signal that acquires information on heartbeat of the user.

3. The system according to claim 2, wherein the Doppler signal analysis unit acquires the spectrum energy by performing a fast Fourier transform on the Doppler signal.

4. The system according to claim 3, wherein the sleep-stage definition unit defines a stage in which the non-periodic spectrum energy does not exist, among stages where the ratio of respiration spectrum energy and heartbeat spectrum energy is 5:5 or higher in the entire sleep stage, as a deep sleep stage, and defines a stage in which the non-periodic spectrum energy does not exist, among stages where the ratio of respiration spectrum energy and heartbeat spectrum energy is lower than 5:5, as an apnea stage.

5. The system according to claim 4, wherein the sleep-stage definition unit defines a stage in which the non-periodic spectrum energy exists and the non-periodic spectrum energy appears to be higher than or equal to a preset magnitude as a tossing and turning stage, and defines a stage in which the non-periodic spectrum energy appears to be lower than or equal to a preset magnitude in the apnea stage as a snoring stage.

6. A sleep state determination system using a biological activity Doppler signal, the system comprising:
a Doppler signal acquisition unit for acquiring a Doppler signal including biological activity information using a radar;
an auxiliary signal processing unit for acquiring a tossing and turning motion and a snoring noise as auxiliary signals using a thermal image sensor and a noise sensor;
a Doppler signal analysis unit for acquiring spectrum energy at preset intervals by analyzing the Doppler signal, determining whether the spectrum energy is acquired periodically, and performing, when the spectrum energy is determined as non-periodic spectrum energy, classification of the non-periodic spectrum energy using the auxiliary signals; and
a sleep-stage definition unit for defining sleep states of the other sleep stages using a preset ratio range and the non-periodic spectrum energy on the basis of an average of the spectrum energy of a sleep entry stage that satisfies preset criteria in an entire sleep stage.

7. A sleep state determination method using a biological activity Doppler signal, the method comprising:
a Doppler signal acquisition step of acquiring, by a Doppler signal acquisition unit, a Doppler signal including biological activity information using a radar;
an auxiliary signal processing step of acquiring, by an auxiliary signal processing unit, a tossing and turning motion and a snoring noise as auxiliary signals using a thermal image sensor and a noise sensor;
a Doppler signal analysis step of acquiring, by a Doppler signal analysis unit, spectrum energy at preset intervals by analyzing the Doppler signal, determining whether the spectrum energy is acquired periodically, and performing, when the spectrum energy is determined as non-periodic spectrum energy, classification of the non-periodic spectrum energy using the auxiliary signals; and
a sleep-stage definition step of defining, by a sleep-stage definition unit, a sleep state of each stage in an entire sleep stage using a combination of a ratio of respiration spectrum energy and heartbeat spectrum energy, among the spectrum energy, and the non-periodic spectrum energy.

8. The method according to claim 7, wherein the Doppler signal includes a respiration Doppler signal that acquires information on breathing of a user and a heartbeat Doppler signal that acquires information on heartbeat of the user.

9. The method according to claim 8, wherein the Doppler signal analysis step acquires the spectrum energy by performing a fast Fourier transform on the Doppler signal.

10. The method according to claim 9, wherein the sleep-stage definition step defines a stage in which the non-periodic spectrum energy does not exist, among stages where the ratio of respiration spectrum energy and heartbeat spectrum energy is 5:5 or higher in the entire sleep stage, as a deep sleep stage, and defines a stage in which the non-periodic spectrum energy does not exist, among stages where the ratio of respiration spectrum energy and heartbeat spectrum energy is lower than 5:5, as an apnea stage.

11. The method according to claim 10, wherein the sleep-stage definition step defines a stage in which the non-periodic spectrum energy exists and the non-periodic spectrum energy appears to be higher than or equal to a preset magnitude as a tossing and turning stage, and defines a stage in which the non-periodic spectrum energy appears to be lower than or equal to a preset magnitude in the apnea stage as a snoring stage.

12. A sleep state determination method using a biological activity Doppler signal, the method comprising the steps of:
acquiring, by a Doppler signal acquisition unit, a Doppler signal including biological activity information using a radar;
acquiring, by an auxiliary signal processing unit, a tossing and turning motion and a snoring noise as auxiliary signals using a thermal image sensor and a noise sensor;
acquiring, by a Doppler signal analysis unit, spectrum energy at preset intervals by analyzing the Doppler signal, determining whether the spectrum energy is acquired periodically, and performing, when the spectrum energy is determined as non-periodic spectrum energy, classification of the non-periodic spectrum energy using the auxiliary signals; and
defining, by a sleep-stage definition unit, sleep states of the other sleep stages using a preset ratio range and the non-periodic spectrum energy on the basis of an average of the spectrum energy of a sleep entry stage that satisfies preset criteria in an entire sleep stage.
